(19) United States
(12) Patent Application Publication
(10) Pub. No.: US 2013/0189789 A1
(43) Pub. Date: Jul. 25, 2013

(54) High Range Activated Clotting Time Assay Formulation

(71) Applicant: Medtronic, Inc., Minneapolis, MN (US)

(72) Inventors: Charlene Yuan, Woodbury, MN (US); Trevor C. Huang, Maple Grove, MN (US); Tessy Kanayinkal, Brooklyn Park, MN (US); Mary Jo Passenheim, Coon Parids, MN (US)

(73) Assignee: MEDTRONIC, INC., Minneapolis, MN (US)

(21) Appl. No.: 13/748,922

(22) Filed: Jan. 24, 2013

Related U.S. Application Data
(60) Provisional application No. 61/590,462, filed on Jan. 25, 2012.

(51) Int. Cl. G01N 33/86

(52) U.S. Cl. CPC G01N 33/86
USPC 436/69; 422/554; 137/15.01

(57) ABSTRACT

High range activated clotting time (HR-ACT) tests detect blood clotting time in blood samples which have high levels of heparin. Reagents such as calcium chloride and kaolin within the test apparatus trigger clotting. Physical separation of the reagents from each other prevents interaction between them before the test sample introduction. In one embodiment, to improve dry kaolin re-suspension, kaolin may be suspended in water rather than buffered saline before being dispensed into the test chamber. The combination of removing the buffered saline and the calcium chloride from the kaolin suspension enables clot detection in blood samples containing 5-6 u/ml of heparin.

### RELATED APPLICATIONS

This application claims the benefit of U.S. Provisional Patent Application 61/590,462 filed Jan. 25, 2012. The disclosures of which are herein incorporated by reference in their entirety.

### TECHNICAL FIELD

This invention relates to detecting changes in viscosity of biologic fluid test samples, e.g., detecting coagulation and coagulation-related activities including agglutination and fibrinolysis of human blood test samples, and more particularly to improved methods and apparatus for obtaining a coagulation time of a blood test sample.

### BACKGROUND

Blood coagulation is a complex chemical and physical reaction that occurs when blood comes into contact with an activating agent, such as an activating surface or an activating agent. (In this context, the term “blood” means whole blood, citrated blood, platelet concentrate, plasma, or control mixtures of plasma and blood cells, unless otherwise specifically called out otherwise; the term particularly includes heparinized blood.)

Several tests of coagulation are routinely utilized to assess the complicated cascade of events leading to blood clot formation and test for the presence of abnormalities or inhibitors of this process. Among these tests are activated clotting time (ACT), which includes high range ACT (HRACT), a test which features a slope response to moderate to high heparin levels in whole blood drawn from a patient during cardiac surgery.

During heart bypass surgery, real-time assessment of clotting function at the operative site is performed to evaluate the result of therapeutic interventions and also to test and optimize, a priori, the treatment choice and dosage.

High Range Activated Clotting Time (HR-ACT) is a test used to monitor the effect of high levels of heparin (up to 6 u/ml) during cardiac pulmonary bypass surgery. HR-ACT tests are based on the viscosity change of a test sample within a test chamber. During a test cycle, a ferromagnetic washer immersed in the test sample is lifted to the top of the test chamber by magnetic force produced by a magnetic field located at the top of the test chamber; the washer is then held at the top of the test chamber for a specific time. After the specified holding time, the washer is then dropped through the test sample via gravity. The increased viscosity due to the clotting of the test sample of blood clotting slows the motion of the washer. Thus, if the time that the washer travels through a specified distance (i.e., the washer “drop time”) is greater than a preset value (the clot detection sensitivity threshold), a clot is detected and an HR-ACT value is reported.

A particular apparatus and method for detecting changes in human blood viscosity based on this principle is disclosed in U.S. Pat. Nos. 5,629,209 and 6,613,286, in which heparinized blood is introduced into a test cartridge through an injection port and fills a blood receiving/dispensing reservoir. The blood then moves from the reservoir through at least one conduit into at least one blood-receiving chamber where it is subjected to a viscosity test. A freely movable ferromagnetic washer is also located within the blood-receiving chamber that is moved up using an electromagnet of the test apparatus and allowed to drop with the force of gravity. Changes in the viscosity of the blood that the ferromagnetic washer falls through are detected by determining the position of the ferromagnetic washer in the blood-receiving chamber over a given time period or a given number of rises and falls of the ferromagnetic washer. The blood sample can be mixed with a viscosity-altering agent (e.g., protamine) as it passes through the conduit to the blood-receiving chamber. Air in the conduit and blood-receiving chamber is vented to atmosphere through a further vent conduit and an air vent/fluid plug as the blood sample is fills the blood-receiving chamber.

The movement of the washer in the above approach is actively controlled only when it is moved up, and the washer passively drops with the force of gravity. The washer is free to float in the test chamber and may drift side-to-side as it is moved up or floats downward. The side-to-side drifting movement may affect the rise time and the fall time, which could add error to the coagulation time measured. The washer may eventually stop moving as a clot forms about it, and no additional information can be obtained on the coagulation process in the sample.

### SUMMARY

It has been discovered that, in a blood sample that is heparinized with high level of heparin, the anticoagulant effect of the heparin requires a higher level of calcium to promote clotting than in conventional tests at lower heparin levels. Conventional tests involve a contact activator, or a mixture of contact activators, such as kaolin, celite and glass beads in a buffered saline solution. Calcium chloride is mixed with the buffered activator suspension solution. The activation reagent is dispensed into the test chamber and then dried (in the dry reagent format). The discovery that the dried kaolin and calcium chloride mixture does not release all the calcium back to the solution after it is mixed with test fluid cannot be addressed by increasing the calcium concentration in the calcium-kaolin mixture. It has been discovered that this approach does not solve the problem because of the interaction between the (positively charged) calcium ions and the (negatively charged) kaolin. Because the calcium ion is not all freed from the kaolin to bind to clotting factors, or to inhibit the anticoagulant effect of the heparin, the dry formulation of kaolin mixed with calcium cannot enable blood samples to clot in the presence of high levels of heparin (5 to 6 u/ml).

It has been further discovered that physical separation of calcium chloride from the kaolin suspension solution—the opposite of the conventional practice of mixing the calcium chloride with kaolin and co-dispensed the together (typically on the bottom of the test chamber—solves the problem. That is, in one embodiment, calcium chloride is dispensed on top of the washer so there is no interaction of calcium ions with kaolin before the test sample introduction.

In another embodiment, to improve dry kaolin re-suspension, kaolin may be suspended in water rather than buffered saline before being dispensed into the test chamber.

The combination of removing the buffered saline and the calcium chloride from the kaolin suspension enables clot detection in blood samples containing 5-6 u/ml of heparin.

Thus, in general terms, an improved cartridge for blood clot detection comprises a test chamber, a first positively charged reagent at a first location within the cartridge, and a second, negatively charged reagent at a second location within the cartridge, such that the first and second reagents are physically separated from each other. In one embodiment, the cartridge further comprises a washer having an upper face which serves as the first portion of the test chamber. In another embodiment, the cartridge further comprises a conduit for introduction of the blood sample into the test chamber, and the first portion of the cartridge is the conduit. In either case, the first reagent may comprise calcium or, independently, the second reagent may comprise kaolin.

An improved method of manufacturing a cartridge for measuring clotting time of a sample of blood introduced into a chamber within the cartridge comprises providing the cartridge with a first location for a first positively charged reagent, and a second location for a second, negatively charged reagent, such that the first and second reagents are physically separated from each other prior to the sample of blood being introduced into the chamber. Again, in one embodiment, the cartridge further comprises a washer having an upper face which serves as the first portion of the test chamber. In another embodiment, the cartridge further comprises a conduit for introduction of the blood sample into the test chamber, and the first portion of the cartridge is the conduit. In either case, the first reagent may comprise calcium or, independently, the second reagent may comprise kaolin.

An improved method of detecting formation of a clot in a blood sample with a washer moving through the sample comprises providing a cartridge defining a test chamber for the sample, the cartridge comprising the washer within the test chamber; providing a first portion of the cartridge with a first positively charged reagent, and a second portion of the cartridge with a second, negatively charged reagent, such that the first and second reagents are physically separated from each other; and introducing the blood sample into the test chamber such that the first and second reagents are mixed into the blood sample. As before, in one embodiment, the cartridge further comprises a washer having an upper face which serves as the first portion of the test chamber. In another embodiment, the cartridge further comprises a conduit for introduction of the blood sample into the test chamber, and the first portion of the cartridge is the conduit. In either case, the first reagent may comprise calcium or, independently, the second reagent may comprise kaolin.

This summary of the claims has been presented here simply to point out some of the ways that the claims overcomes difficulties presented in the prior art and to distinguish the claims from the prior art and is not intended to operate in any manner as a limitation on the interpretation of claims that are presented initially in the patent application and that are ultimately granted.

### BRIEF DESCRIPTION OF THE DRAWINGS

These and other advantages and features will be more readily understood from the following detailed description of various embodiments, when considered in conjunction with the drawings, in which like reference numerals indicate identical structures throughout the several views, and in which:

FIG. 1, which is based on FIG. 13 of U.S. Pat. No. 5,629,209, is a cross-sectional view of a cartridge positioned within a machine.

FIG. 2, which is based on FIG. 12d of U.S. Pat. No. 5,629,209, is a partial cross-sectional view of the cartridge of FIG. 1.

FIG. 3 is a schematic cross-section of a first embodiment of the test chamber portion of the cartridge of FIGS. 1 and 2.

FIG. 4 is a schematic cross-section of a second embodiment of the test chamber portion of the cartridge of FIGS. 1 and 2.

FIG. 5 is a graph of data from comparative activated clot time (ACT) tests according to various embodiments.

### DETAILED DESCRIPTION

In the following detailed description, references are made to illustrative embodiments of methods and apparatus for carrying out the claims. It is understood that other embodiments can be utilized without departing from the scope of the claims. Exemplary methods and apparatus are described for performing blood coagulation tests of the type described above.

FIG. 1 only illustrates the basic features of a suitable apparatus, as known from U.S. Pat. No. 5,629,209, the entirety of which is incorporated by reference. The cartridge 100, having been inserted into the side 16 of the machine 10, is secured within the cartridge holder 302. An aperture 28 enables the fluid sample to be introduced into the cartridge 100 after the cartridge 100 is inserted into the machine 10. An air vent/fluid plug device 120 is aligned over a hole 304 in the base of the cartridge holder 302 to permit escape of air that is vented from the cartridge 100 during the movement of the fluid sample into its respective fluid-receiving chamber. Each fluid-receiving chamber may be associated with a means for moving the ferromagnetic material (e.g., a washer made of a ferromagnetic material) provided by the machine 10, such as an electromagnet 122, and a means for detecting the position of the ferromagnetic material 116 within the chamber 114, e.g., a detector 124. A radio frequency detector may be conveniently employed for this purpose. It should be noted that the detector 124 is not limited to the detection of ferromagnetic material but is capable of detecting any metallic substance placed within the chamber 114. The electromagnet 122 and the position detector 124 are connected to a circuit board 300 through which an associated computer receives information, provides directions, and provides test results. For simplicity of illustration, only one fluid-receiving chamber 114, electromagnet 122, and position detector 124 are shown. Cartridge 100 may have a plurality of such arrangements for alternative and/or comparative tests.

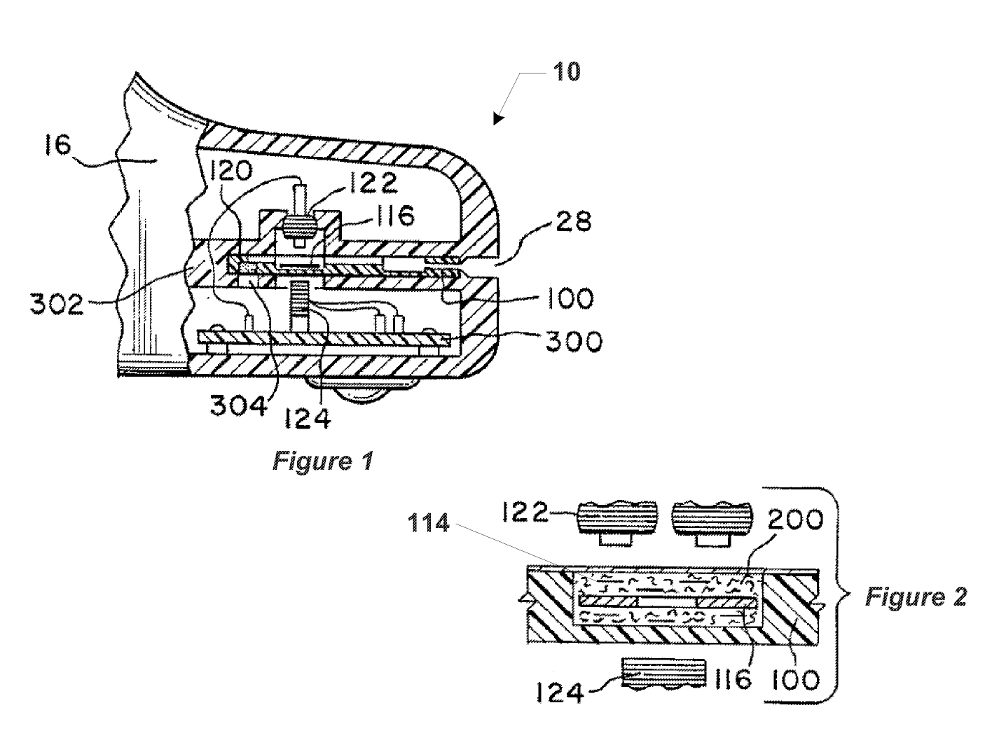

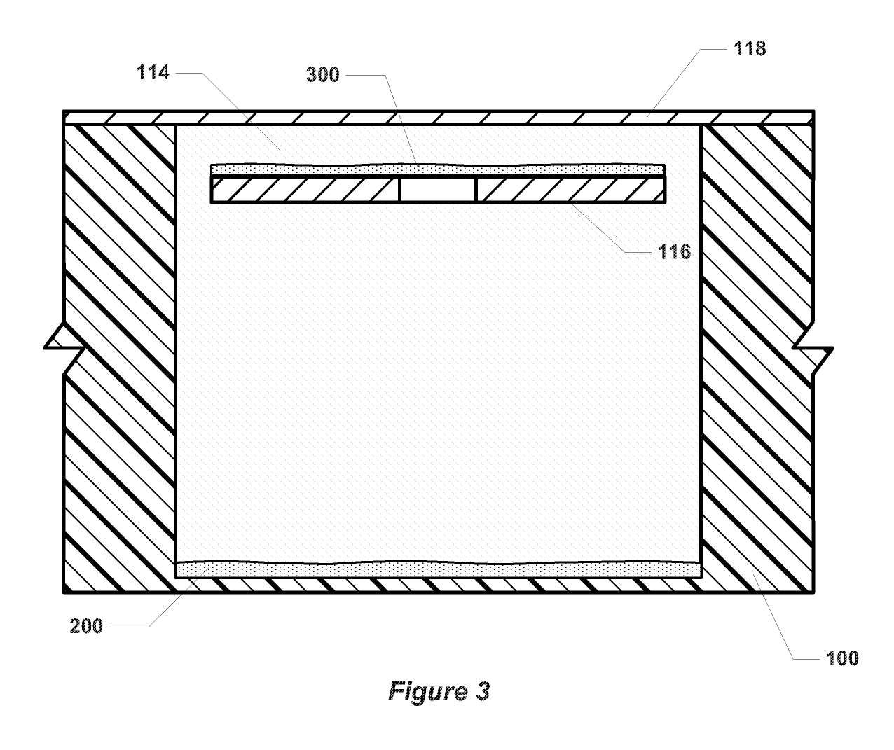

FIG. 2 illustrates that fluid 200 fills the fluid-receiving chamber and reaches the air vent/fluid plug device 120 to establish a fluid lock. Ferromagnetic washer 116 is moved between a resting position on the bottom of the fluid-receiving chamber 114 and the top of the chamber 114 as the electromagnet 122 is energized; if the electromagnet 122 is turned off the washer 116, under the force of gravity, falls through the fluid 200 to the bottom of the chamber 114. The position detector 124 measures the time required for the washer 116 to fall from the top to the bottom of the chamber 114 and sends this information to the associated computer. As the viscosity of the fluid 200 increases, the measured time increases. Indeed, in the case of blood coagulation, eventually, a washer 116 is unable to move through a blood sample.

When the fluid 200 whose viscosity is being measured is blood, the motion of the washer 116 through the blood also has the effect of activating the clotting process of the blood. The activation effect is enhanced when the surface of the washer 116 is roughened in known ways, as such techniques increase the surface area of the washer. If even faster clotting times are necessary, a viscosity-altering substance may be used. For example, a clotting activator such as tissue thromboplastin can be added to the cartridge, or a particulate activator such as diatomaceous earth or kaolin may be used either alone or in combination with a viscosity-altering substance such as protamine or thromboplastin.

The position detector 124 may be a radio frequency detector. Radio frequency detectors sense the position of the washer 116 by sensing the changes in the magnetic field surrounding the detection coil of the radio frequency detector that are caused by the presence of the washer 116. Radio frequency detectors also are sensitive to ferromagnetic and other metallic materials and resistance to effects caused by other elements of the device, such as the fluid. It should be understood, however, that other types of position detectors 124 are contemplated. For example, in another embodiment, the position detector 124 is a Hall effect sensor and its associated circuitry, as generally described in U.S. Pat. No. 7,775,976 (the entirety of which is incorporated by reference) at column 16, line 15 to column 17, line 5. Regardless of the type of position detector 124 employed, the absolute position of the washer 116 is measured and used as described below.

In a typical sequence, a sample mix cycle begins the test protocol. The electromagnet 122 initially raises and lowers the washer 116 rapidly several times to further mix the fluid 200 with any viscosity-altering substance present and, if the fluid 200 is blood, promote activation of clotting, as discussed above. The fluid 200 is then allowed to rest for a short time. During the subsequent test itself, the electromagnet 122 raises the washer 116 repeatedly at a slower rate. After each elevation of the washer, the position detector 124 is used to determine the “fall time” (or “drop time”), i.e., the time taken for the washer 116 to fall to the bottom of the chamber 114. Absence of an increase in fall time suggests a lack of coagulation and the test continues. But an increase in fall time suggests a change in viscosity, measured in terms of the amount of fall time as compared to a baseline value. All data, including individual test results, may be displayed, stored in memory, printed, or sent to another computer, or any combination of the same.

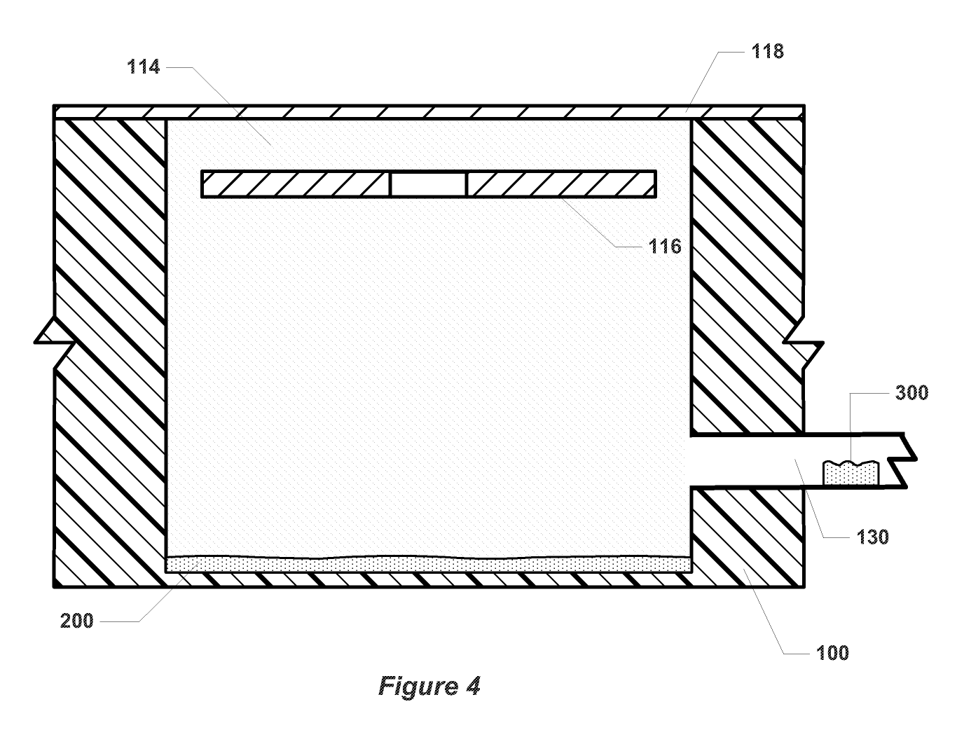

The principles of the first embodiment are schematically illustrated in FIG. 3. The electromagnet 122, position detector 124, and fluid 200 have been omitted for clarity only. Similarly, the height of the chamber 114 is exaggerated relative to the thickness of the washer 116 only for purposes of illustration.

Calcium chloride composition 300 is physically separated from kaolin composition 200 within the test chamber; that is, the two compositions do not touch each other prior to introduction of the fluid sample. In the embodiment illustrated, this physical separation is ensured by providing the kaolin composition 200 at a first location, such as a portion of the interior surfaces of the chamber itself, such as the bottom of the chamber as illustrated; and then providing the calcium chloride composition 300 at a second, different location such as a surface of the washer 116, such as the upper surface as illustrated. Thus, the washer 116 is a physical barrier which ensures separation of the calcium chloride composition 300 from the kaolin composition 200 during the manufacture and storage of cartridge 100. When the cartridge 100 is used in testing, the blood specimen will dissolve the calcium chloride composition 300 on the washer and only then will the dissolved calcium chloride composition 300 mix with the kaolin composition 200 (which will also be re-suspended by the blood specimen) on the bottom of the well. Together, the calcium chloride composition 300 and the kaolin composition 200 will activate the blood specimen and initiate the clotting process.

A second embodiment is illustrated in FIG. 4, in which portions of the second embodiment which are common to the first embodiment are numbered as in FIG. 3 and need no additional explanation. A “runner” or conduit 130 through which the fluid sample is introduced into the test chamber 114 is explicitly shown. Such a conduit is conventional and was omitted from FIG. 3 only for clarity. The location, dimensions, and configuration of the conduit 130 are schematic only.

The second embodiment addresses two possible problems presented by the embodiment of FIG. 3. The first problem is that the calcium chloride composition 300 on the upper face of the washer 116 may stick to the cartridge cover 118 during cartridge self-testing cycles. During these cycles, the washer 116 is elevated without any fluid sample in the chamber 114. It is desirable to avoid cartridge failures caused by such sticking The second possible problem is that there may be interactions between the (positively charged) calcium ions and the metal material of washer 116, particularly if the shelf life of a cartridge is long.

To avoid these problems, the second embodiment places the calcium chloride composition 300 within runner 130 at a location such that calcium chloride composition 300 remains physically separated from the kaolin composition 200, as by a gap between the two compositions. As in the first embodiment, the blood sample will dissolve the calcium chloride composition 300 during the sample fill process and mix it with the kaolin reagent 200. The calcium chloride composition 300 will continue to be mixed into the kaolin during the test cycles, because the movement of washer 116 during the test cycles will draw fluid from the nearby runner 130.

In both of the embodiments above, the amounts of kaolin composition 200 and calcium chloride composition 300 used are determined in accordance with known principles and not affected by their physical separation from each other.

### EXAMPLE 1

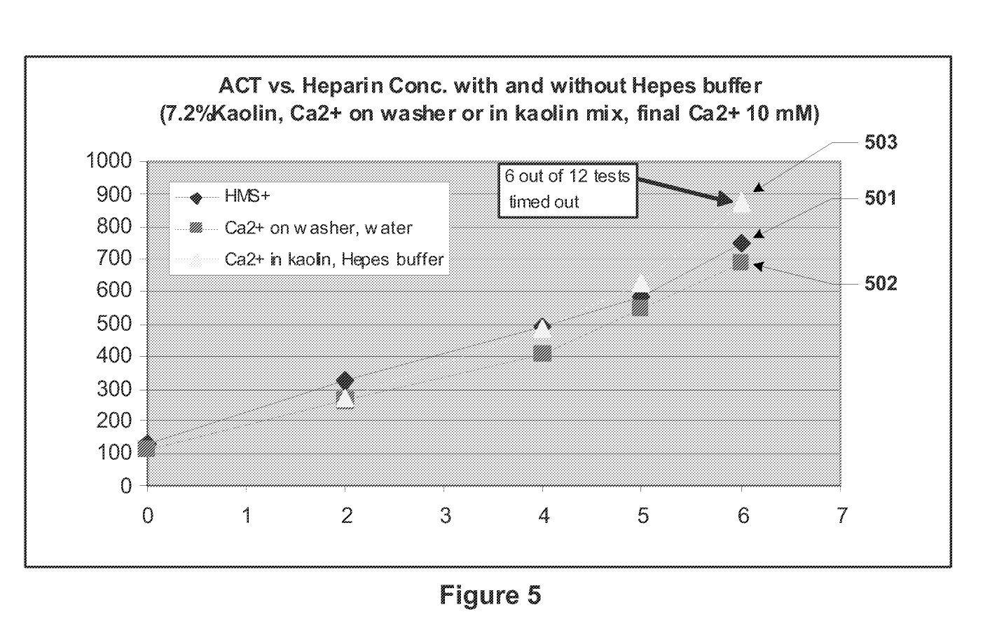

FIG. 5 shows a comparison of results from a cartridge made as described in U.S. Pat. No. 6,613,286 and having different compositions. The graph is time to detect a clot (seconds) as a function of heparin concentration (u/ml).

In the control, the calcium ions were mixed with the kaolin reagent as done conventionally. In the two tests, the calcium ions were provided on the washer, either mixed with water alone or a buffered solution of HEPES [4-(2-hydroxyethyl)-1-piperazineethanesulfonic acid]. All tests were performed with the blood of five donors.

On average, the calcium chloride composition 300 removed from the kaolin reagent gave lower ACT values 502 compared to the control 501; the calcium chloride composition 300 mixed with dry kaolin reagent in the sample well gave higher ACT values 503 than the control at high heparin concentrations (5 and 6 u/ml). With the formulation of calcium ions in kaolin, three out of five donor samples timed out (no clotting detected after 1000 seconds) at 6 u/ml heparin concentration. This experiment suggests that removal of calcium ions from the dry kaolin mixture helps the cartridge detect high levels of heparin.

While the description above uses the apparatus and procedures of U.S. Pat. Nos. 5,629,209 and 6,613,286 to describe certain details, the broadest scope of the disclosure includes any apparatus which relies on any combination of analog or digital hardware, as well as methods of manufacturing or using the same, that do not depend upon the specific physical components mentioned above but nonetheless achieve the same or equivalent results. Therefore, the full scope of the invention is described by the following claims.

What is claimed is:

1. A cartridge for blood clot detection, comprising a test chamber, a first positively charged reagent at a first location within the cartridge, and a second, negatively charged reagent at a second location within the cartridge, such that the first and second reagents are physically separated from each other.

2. The cartridge of claim 1, in which the cartridge further comprises a washer having an upper face, and the first portion of the test chamber is the upper face of the washer.

3. The cartridge of claim 1, in which the cartridge further comprises a conduit for introduction of the blood sample into the test chamber, and the first portion of the cartridge is the conduit.

4. The cartridge of claim 1, in which the first reagent comprises calcium.

5. The cartridge of claim 1, in which the second reagent comprises kaolin.

6. A method of manufacturing a cartridge for measuring clotting time of a sample of blood introduced into a chamber within the cartridge, comprising providing the cartridge with a first location for a first positively charged reagent and a second location for a second, negatively charged reagent, such that the first and second reagents are physically separated from each other prior to the sample of blood being introduced into the chamber.

7. The method of claim 6, further comprising providing the cartridge with a washer having an upper face, in which the first portion of the test chamber is the upper face of the washer.

8. The method of claim 6, in which the cartridge further comprises a conduit for introduction of the blood sample into the test chamber, and the first portion of the cartridge is the conduit.

9. The method of claim 6, in which the first reagent comprises calcium.

10. The method of claim 6, in which the second reagent comprises kaolin.

11. A method of detecting formation of a clot in a blood sample with a washer moving through the sample, comprising:
a. providing a cartridge defining a test chamber for the sample, the cartridge comprising the washer within the test chamber;
b. providing a first portion of the cartridge with a first positively charged reagent, and a second portion of the cartridge with a second, negatively charged reagent, such that the first and second reagents are physically separated from each other; and
c. introducing the blood sample into the test chamber such that the first and second reagents are mixed into the blood sample.

12. The method of claim 11, in which the washer has an upper face and the first portion of the test chamber is the upper face of the washer.

13. The method of claim 11, in which the cartridge further comprises a conduit for introduction of the blood sample into the test chamber, and the first portion of the cartridge is the conduit.

14. The method of claim 11, in which the first reagent comprises calcium.

15. The method of claim 11, in which the second reagent comprises kaolin.
